(19) United States
(12) Patent Application Publication
(10) Pub. No.: US 2020/0363410 A1
(43) Pub. Date: Nov. 19, 2020

(54) A PROCESS FOR IMMOBILIZING POLYPEPTIDES

(71) Applicant: UNIVERSITY OF DELHI SOUTH CAMPUS, New Delhi (IN)

(72) Inventors: Vijay Kumar CHAUDHARY, New Delhi (IN); Amita GUPTA, New Delhi (IN); Vaishali VERMA, New Delhi (IN); Charanpreet KAUR, New Delhi (IN); Payal GROVER, New Delhi (IN)

(21) Appl. No.: 16/762,770

(22) PCT Filed: Nov. 8, 2018
(86) PCT No.: PCT/IN2018/050722

§ 371 Date: May 8, 2020

(30) Foreign Application Priority Data
Nov. 9, 2017 (IN) 201711040047

(51) Int. Cl. G01N 33/543; G01N 33/68; C07K 16/12

(52) U.S. Cl. CPC G01N 33/54393; G01N 2333/35; C07K 16/1289; G01N 33/6854

(57) ABSTRACT

The present disclosure discloses a process for immobilizing polypeptides on a surface, said method comprising: (a) coating a surface with a molecule to capture biotin tagged polypeptide to obtain a coated surface; and (b) contacting at least two biotin tagged polypeptides with the coated surface of step (a) to obtain immobilized polypeptides, wherein the biotin tagged polypeptide comprises a biotin linked to a recombinant polypeptide. The present disclosure further discloses an in-vitro method for detecting at least one binder molecule in a sample, and a process for obtaining biotin tagged polypeptide.

### FIELD OF INVENTION

The present disclosure relates to the field of recombinant polypeptide engineering in general and a process of immobilizing biotin tagged polypeptides onto a coated surface in particular.

### BACKGROUND OF THE INVENTION

Immunoassays are the means of detection and diagnosis of various infectious diseases and exploits the specific interaction between an antigen and an antibody. Principally, these methods are based on a competitive binding reaction between a fixed amount of labelled form of an analyte and a variable amount of unlabelled sample analyte for a limited number of binding sites on a highly specific anti-analyte antibody (Darwish, Int J Biomed Sci, 2006, 2(3): 217-235).

Under conventional approaches, immunoassays in general employ immobilization of an analyte or protein onto a polystyrene surface. The immobilization is a passive adsorption of the protein and primarily relies on hydrophobic interactions. Such passive adsorption of the proteins is not only non-specific, but also leads undesirable denaturation of protein which in turn makes the protein undetectable or unrecognizable to the specific binders. Furthermore, passive immobilization of proteins may be highly random and variable depending on the composition and exposure of hydrophobic patches of proteins, thereby causing non-uniformity in the coating of proteins (Schetters H. Biomolecular engineering. 1999; 16(1-4):73-8).

In case of infectious diseases such as tuberculosis, there is variability in the antibody profiles from one patient to another and therefore detection by means of passive adsorption of antigens which is random and non-uniform, is of little or no consequence.

The high affinity interaction between streptavidin and biotin has enabled specific and efficient capture of proteins tagged with biotin on either of the terminus, onto a streptavidin coated surface. Such capture is much more directed and specific in nature as compared to passive adsorption. The interaction has shown to increase the sensitivity of detection of anti-EPO antibodies in human sera by using biotinylated rhEPO coating onto streptavidin coated microtiter plates (Gross. et.al. Journal of Immunological Methods, 2006, 313(1-2):176-182)

Though, studies have been performed with respect to detection of antibodies using single biotinylated antigens, such process is of little consequence when detecting diverse antibodies in case of diseases such as tuberculosis. Therefore, there is a need of developing processes that would allow early and efficient detection of such infectious diseases.

### SUMMARY OF INVENTION

In an aspect of the present disclosure, there is provided a method of immobilizing polypeptides on a surface, said method comprising: (a) coating a surface with a molecule to capture biotin tagged polypeptide to obtain a coated surface; and (b) contacting at least two biotin tagged polypeptides with the coated surface of step (a) to obtain immobilized polypeptides, wherein the biotin tagged polypeptide comprises biotin linked to a recombinant polypeptide.

In an aspect of the present disclosure, there is provided an in-vitro method for detecting at least one binder in a sample, said method comprising: (a) coating a surface with a molecule to capture biotin tagged polypeptide to obtain a coated surface; (b) contacting at least two biotin tagged polypeptides with the coated surface of step (a) to obtain immobilized polypeptides, wherein the biotin tagged polypeptide comprises biotin linked to a recombinant polypeptide; (c) obtaining a sample; (d) adding the sample to the immobilized polypeptides to obtain a binder-polypeptide complex; and (e) detecting the binder-polypeptide complex, wherein detecting the binder-polypeptide complex indicates the presence of at least one binder in the sample.

In an aspect of the present disclosure, there is provided a biotin tagged polypeptide comprising a recombinant polypeptide linked to a biotin, wherein the recombinant polypeptide has an amino acid sequence selected from a group consisting of SEQ ID NO: 1, SEQ ID NO: 3, SEQ ID NO: 5, SEQ ID NO: 7, and SEQ ID NO: 9.

In an aspect of the present disclosure, there is provided a recombinant nucleic acid molecule having nucleotide sequence selected from a group consisting of SEQ ID NO. 2, SEQ ID NO. 4, SEQ ID NO. 6, SEQ ID NO. 8, and SEQ ID NO. 10.

In an aspect of the present disclosure, there is provided a recombinant vector comprising a recombinant nucleic acid molecule having nucleotide sequence selected from a group consisting of SEQ ID NO. 2, SEQ ID NO. 4, SEQ ID NO. 6, SEQ ID NO. 8, and SEQ ID NO. 10, operably linked to a promoter to drive the expression of the recombinant nucleic acid molecule.

In an aspect of the present disclosure, there is provided a recombinant host cell comprising a vector as described herein above.

In an aspect of the present disclosure, there is provided a process for expression of a recombinant polypeptide, said process comprising the steps of: (a) obtaining a recombinant host cell as described herein above; (b) growing the recombinant host cell in a growth medium under suitable conditions for the expression of the recombinant polypeptide.

In an aspect of the present disclosure, there is provided a process of preparing a biotin tagged polypeptide, said process comprising the steps of: (a) obtaining a recombinant host cell as described herein above; (b) growing the recombinant host cell in a growth medium under suitable conditions to express a recombinant polypeptide, wherein the recombinant polypeptide comprises a histidine affinity tag, a TEV protease site, and a BAP tag having amino acid sequence as depicted in SEQ ID NO: 11; (c) contacting the recombinant polypeptide of step (b) with an affinity chromatographic support; (d) eluting a polypeptide fraction 1 from the affinity chromatographic support; wherein the polypeptide fraction 1 has a histidine affinity tag; (e) contacting the polypeptide fraction 1 with a gel filtration chromatographic support; (f) eluting a polypeptide fraction 2 from the gel filtration chromatographic support, wherein the polypeptide fraction 2 has a histidine affinity tag; (g) treating the polypeptide fraction 2 with tagged TEV protease to remove the affinity tag from the polypeptide fraction 2 to obtain a polypeptide fraction 3; (h) contacting the polypeptide fraction 3 with an affinity chromatographic support; (i) eluting a polypeptide fraction 4 from the affinity chromatographic support, wherein the polypeptide fraction 4 does not have a histidine affinity tag; (j) contacting the polypeptide fraction 4 with an anion-exchange chromatographic support; (k) eluting the recombinant polypeptide from the anion-exchange chromatographic support; (l) enzymatically linking the recombinant polypeptide of step (k) to a biotin molecule using histidine tagged recombinant BirA enzyme; and (m) removing the histidine tagged recombinant BirA enzyme from enzymatically linked recombinant polypeptide of step (l) by affinity chromatography to obtain the biotin tagged polypeptide.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 shows the vector map for pVMExp14367, in accordance with an embodiment of the present disclosure.

FIG. 2 shows an SDS gel image depicting the expression profile of the recombinant MTC28, MPT63, MPT64, Ag85A, and Ag85B in accordance with an embodiment of the present disclosure.

FIG. 3 shows an SDS gel image depicting purification profile of MTC28 polypeptide, in accordance with an embodiment of the present disclosure.

FIG. 4 shows an SDS gel image depicting purification profile of MPT63 polypeptide, in accordance with an embodiment of the present disclosure.

FIG. 5 shows an SDS gel image depicting purification profile of MPT64 polypeptide, in accordance with an embodiment of the present disclosure.

FIG. 6 shows an SDS gel image depicting purification profile of Ag85A polypeptide, in accordance with an embodiment of the present disclosure.

FIG. 7 shows an SDS gel image depicting purification profile of Ag85B polypeptide, in accordance with an embodiment of the present disclosure.

FIG. 8 shows an SDS gel image depicting purified biotinylated MTC28, MPT63, MPT64, Ag85A, and Ag85B polypeptide, in accordance with an embodiment of the present disclosure.

FIG. 9A to 9D depicts results for ELISA using biotinylated MTC28, MPT63, MPT64, Ag85A, and Ag85B polypeptide, in accordance with an embodiment of the present disclosure.

FIG. 10 provides a simplified workflow for expression, purification, and in vitro biotinylation of the recombinant polypeptides, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

For the purposes of the present disclosure, the term ‘binder’ refers to specific molecule which has binding affinity to the corresponding biotinylated polypeptide. It may refer to a corresponding antigen or antibody. It may also refer to any other polypeptide or nucleic acid molecule having affinity to the corresponding biotinylated polypeptide.

For the purposes of the present disclosure, the term ‘surface’ refers to a support matrix selected from a group consisting of glass and activated glass surface, silanized glass surface, silicon surface, maleimide or maleic anhydride-coated surface, polybrene-coated surface, metal surface, paramagnetic surface, gold-coated surface, immobilized metal-affinity surface, plastic surface referring to polystyrene, polypropylene, polyethylene, poly(methylmethacrylate), polydimethysiloxane, plasma polymers including allylamine, cyclopropylamine, bromine, polyethylene glycol (PEG), diethylene glycol dimethyl ether (diglyme), cyclic polyolefin (COP), silanized, dextran-coated surface, activated surfaces with carboxyl, hydroxyl, thiol, amine, aldehyde groups, or carrying glycidoxy, thiocyano, isocyanate, succinimidyloxy- or succinimidyl ester, aryl azides/azido groups, hydrazine/hydrazide, alkyl halide, benzyl halide, α-halo acetyl reactive groups, paper, membrane including nitrocellulose, PVDF, nylon, and latex.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background section, detection of infectious diseases caused by organisms such as Mycobacterium spp, Pseudomonas spp, and the likes is challenging owing to the variable profile of the antibodies that are generated in the body in response to these organisms. Detection of such diseases is often difficult and inconclusive with conventional approaches of detection which employs use of only a single type of antigen. Additionally, such conventional approach utilizes passive adsorption of antigen onto a polystyrene surface, which more often leads to random coating of antigens and also results in denaturation of proteins. As a result, the sample required for detection is higher and the sensitivity is extremely low.

The present invention provides a solution for the shortcomings in the conventional immunoassay methods as stated above. Provided herein, is a process of immobilizing multiple biotin-tagged polypeptides onto a coated surface which allows uniform binding of all the polypeptides. The method allows detection of antibodies, both monoclonal and polyclonal in samples, such as human sera. It has been further shown that, owing to the uniform immobilization of the biotin-tagged polypeptides onto a streptavidin surface in the present method, the amount of sample required is low and the sensitivity achieved is much higher as compared to passive adsorption of polypeptides. This has further been evidenced by preparing biotin tagged Mycobacterium antigens, more specifically, H37Rv secretory proteins viz. MTC28 (Rv0040c), MPT63 (Rv1926c), MPT64 (Rv1980c), Ag85A (Rv3804c), and Ag85B (Rv1886c). Provided herein is a streamlined workflow for cloning, soluble expression, and purification of the expressed protein, followed by its in vitro biotinylation to obtain highly pure recombinant protein carrying single biotin attached to the lysine residue within the 15-amino acid BAP tag present at the C-terminus. A T7 promoter-lac operator-based IPTG/lactose inducible vector system employing rapid and high-throughput restriction enzyme-free cloning of genes has been developed for auto-induction-based cytosolic expression of recombinant proteins carrying N-terminal deca-histidine tag (H10) followed by TEV protease site, and C-terminal Biotin Acceptor Peptide (BAP) tag with appropriate glycine-serine rich spacers between different elements. Furthermore, a robust three-step chromatography pipeline integrated with well-optimized and highly efficient protocols for TEV protease-based H10 tag removal, and recombinant E. coli BirA enzyme-based site-specific in vitro biotinylation has been described to obtain highly purified and tagless (devoid of the N-terminus H10 tag) proteins carrying single biotin residue at the C-terminus. Most importantly, the utility of these biotin-tagged recombinant proteins has been exemplified by comparison of passive versus specific protein immobilization in the context of indirect ELISA.

Sequence Listing:
depicts recombinant MTC28 amino acid sequence
SEQ ID NO: 1
GASGSDPLLPPPPIPAPVSAPATVPPVQNLTALPGGSSNRFSPAPAPAPI
ASPIPVGAPGSTAVPPLPPPVTPAISGTLRDHLREKGVKLEAQRPHGFKA
LDITLPMPPRWTQVPDPNVPDAFVVIADRLGNSVYTSNAQLVVYRLIGDF
DPAEAITHGYIDSQKLLAWQTTNASMANFDGFPSSIIEGTYRENDMTLNT
SRRHVIATSGADKYLVSLSVTTALSQAVTDGPATDAIVNGFQVVAHAAPA
QAPAPAPGSAPVGLPGQAPGYPPAGTLTPVPPRGGGASGGAPGGLNDIFE
AQKIEWHE
depicts recombinant MTC28 nucleotide sequence
SEQ ID NO: 2
GGTGCTAGCGGCAGCGATCCCCTGCTGCCACCGCCGCCTATCCCTGCCCC
AGTCTCGGCGCCGGCAACAGTCCCGCCCGTGCAGAACCTCACGGCGCTTC
CGGGCGGGAGCAGCAACAGGTTCTCACCGGCGCCAGCACCCGCACCGATC
GCGTCGCCGATTCCGGTCGGAGCACCCGGGTCCACCGCTGTGCCCCCGCT
GCCGCCGCCAGTGACTCCCGCGATCAGCGGCACACTTCGGGACCACCTCC
GGGAGAAGGGCGTCAAGCTGGAGGCACAGCGACCGCACGGATTCAAGGCG
CTCGACATCACACTGCCCATGCCGCCGCGCTGGACTCAGGTGCCCGACCC
CAACGTGCCCGACGCGTTCGTGGTGATCGCCGACCGGTTGGGCAACAGCG
TCTACACGTCGAATGCGCAGCTGGTAGTGTATAGGCTGATCGGTGACTTC
GATCCCGCTGAGGCCATCACACACGGCTACATTGACAGCCAGAAATTGCT
CGCATGGCAGACCACAAACGCCTCGATGGCCAATTTCGACGGCTTTCCGT
CATCAATCATCGAGGGCACCTACCGCGAAAACGACATGACCCTCAACACC
TCCCGGCGCCACGTCATCGCCACCTCCGGAGCCGACAAGTACCTGGTTTC
GCTGTCGGTGACCACCGCGCTGTCGCAGGCGGTCACCGACGGGCCGGCCA
CCGATGCGATTGTCAACGGATTCCAAGTGGTTGCGCATGCGGCGCCCGCT
CAGGCGCCTGCCCCGGCACCCGGTTCGGCACCGGTGGGACTACCCGGGCA
GGCGCCTGGGTATCCGCCCGCGGGCACCCTGACACCAGTCCCGCCGCGCG
GTGGAGGCGCCTCAGGCGGCGCGCCTGGAGGTCTGAACGACATCTTCGAG
GCTCAGAAAATCGAATGGCACGAG
depicts recombinant MPT63 amino acid sequence
SEQ ID NO: 3
GASGSAYPITGKLGSELTMTDTVGQVVLGWKVSDLKSSTAVIPGYPVAGQ
VWEATATVNAIRGSVTPAVSQFNARTADGINYRVLWQAAGPDTISGATIP
QGEQSTGKIYFDVTGPSPTIVAMNNGMEDLLIWEPGGGASGGAPGGLNDI
FEAQKIEWHE
depicts recombinant MPT63 nucleotide sequence
SEQ ID NO: 4
GGTGCTAGCGGCAGCGCCTATCCCATCACCGGAAAACTTGGCAGTGAGCT
AACGATGACCGACACCGTTGGCCAAGTCGTGCTCGGCTGGAAGGTCAGTG
ATCTCAAATCCAGCACGGCAGTCATCCCCGGCTATCCGGTGGCCGGCCAG
GTCTGGGAGGCCACTGCCACGGTCAATGCGATTCGCGGCAGCGTCACGCC
CGCGGTCTCGCAGTTCAATGCCCGCACCGCCGACGGCATCAACTACCGGG
TGCTGTGGCAAGCCGCGGGCCCCGACACCATTAGCGGAGCCACTATCCCC
CAAGGCGAACAATCGACCGGCAAAATCTACTTCGATGTCACCGGCCCATC
GCCAACCATCGTCGCGATGAACAACGGCATGGAGGATCTGCTGATTTGGG
AGCCGGGTGGAGGCGCCTCAGGCGGCGCGCCTGGAGGTCTGAACGACATC
TTCGAGGCTCAGAAAATCGAATGGCACGAG
depicts recombinant MPT64 amino acid sequence
SEQ ID NO: 5
GASGSAPKTYCEELKGTDTGQACQIQMSDPAYNINISLPSYYPDQKSLEN
YIAQTRDKFLSAATSSTPREAPYELNITSATYQSAIPPRGTQAVVLKVYQ
NAGGTHPTTTYKAFDWDQAYRKPITYDTLWQADTDPLPVVFPIVQGELSK
QTGQQVSIAPNAGLDPVNYQNFAVTNDGVIFFFNPGELLPEAAGPTQVLV
PRSAIDSMLAGGGASGGAPGGLNDIFEAQKIEWHE
depicts recombinant MPT64 nucleotide sequence
SEQ ID NO: 6
GGTGCTAGCGGCAGCGCGCCCAAGACCTACTGCGAGGAGTTGAAAGGCAC
CGATACCGGCCAGGCGTGCCAGATTCAAATGTCCGACCCGGCCTACAACA
TCAACATCAGCCTGCCCAGTTACTACCCCGACCAGAAGTCGCTGGAAAAT
TACATCGCCCAGACGCGCGACAAGTTCCTCAGCGCGGCCACATCGTCCAC
TCCACGCGAAGCCCCCTACGAATTGAATATCACCTCGGCCACATACCAGT
CCGCGATACCGCCGCGTGGTACGCAGGCCGTGGTGCTCAAGGTCTACCAG
AACGCCGGCGGCACGCACCCAACGACCACGTACAAGGCCTTCGATTGGGA
CCAGGCCTATCGCAAGCCAATCACCTATGACACGCTGTGGCAGGCTGACA
CCGATCCGCTGCCAGTCGTCTTCCCCATTGTGCAAGGTGAACTGAGCAAG
CAGACCGGACAACAGGTATCGATAGCGCCGAATGCCGGCTTGGACCCGGT
GAATTATCAGAACTTCGCAGTCACGAACGACGGGGTGATTTTCTTCTTCA
ACCCGGGGGAGTTGCTGCCCGAAGCAGCCGGCCCAACCCAGGTATTGGTC
CCACGTTCCGCGATCGACTCGATGCTGGCCGGTGGAGGCGCCTCAGGCGG
CGCGCCTGGAGGTCTGAACGACATCTTCGAGGCTCAGAAAATCGAATGGC
ACGAG
depicts recombinant Ag85A amino acid sequence
SEQ ID NO: 7
GASGSFSRPGLPVEYLQVPSPSMGRDIKVQFQSGGANSPALYLLDGLRAQ
DDFSGWDINTPAFEWYDQSGLSVVMPVGGQSSFYSDWYQPACGKAGCQTY
KWETFLTSELPGWLQANRHVKPTGSAVVGLSMAASSALTLAIYHPQQFVY
AGAMSGLLDPSQAMGPTLIGLAMGDAGGYKASDMWGPKEDPAWQRNDPLL
NVGKLIANNTRVWVYCGNGKPSDLGGNNLPAKFLEGFVRTSNIKFQDAYN
AGGGHNGVFDFPDSGTHSWEYWGAQLNAMKPDLQRALGATPNTGPAPQGA
GGGASGGAPGGLNDIFEAQKIEWHE
depicts recombinant Ag85A nucleotide sequence
SEQ ID NO: 8
GGTGCTAGCGGCAGCTTTTCCCGGCCGGGCTTGCCGGTGGAGTACCTGCA
GGTGCCGTCGCCGTCGATGGGCCGTGACATCAAGGTCCAATTCCAAAGTG
GTGGTGCCAACTCGCCCGCCCTGTACCTGCTCGACGGCCTGCGCGCGCAG
GACGACTTCAGCGGCTGGGACATCAACACCCCGGCGTTCGAGTGGTACGA
CCAGTCGGGCCTGTCGGTGGTCATGCCGGTGGGTGGCCAGTCAAGCTTCT
ACTCCGACTGGTACCAGCCCGCCTGCGGCAAGGCCGGTTGCCAGACTTAC
AAGTGGGAGACCTTCCTGACCAGCGAGCTGCCGGGGTGGCTGCAGGCCAA
CAGGCACGTCAAGCCCACCGGAAGCGCCGTCGTCGGTCTTTCGATGGCTG
CTTCTTCGGCGCTGACGCTGGCGATCTATCACCCCCAGCAGTTCGTCTAC
GCGGGAGCGATGTCGGGCCTGTTGGACCCCTCCCAGGCGATGGGTCCCAC
CCTGATCGGCCTGGCGATGGGTGACGCTGGCGGCTACAAGGCCTCCGACA
TGTGGGGCCCGAAGGAGGACCCGGCGTGGCAGCGCAACGACCCGCTGTTG
AACGTCGGGAAGCTGATCGCCAACAACACCCGCGTCTGGGTGTACTGCGG
CAACGGCAAGCCGTCGGATCTGGGTGGCAACAACCTGCCGGCCAAGTTCC
TCGAGGGCTTCGTGCGGACCAGCAACATCAAGTTCCAAGACGCCTACAAC
GCCGGTGGCGGCCACAACGGCGTGTTCGACTTCCCGGACAGCGGTACGCA
CAGCTGGGAGTACTGGGGCGCGCAGCTCAACGCTATGAAGCCCGACCTGC
AACGGGCACTGGGTGCCACGCCCAACACCGGGCCCGCGCCCCAGGGCGCC
GGTGGAGGCGCCTCAGGCGGCGCGCCTGGAGGTCTGAACGACATCTTCGA
GGCTCAGAAAATCGAATGGCACGAG
depicts recombinant Ag85B amino acid sequence
SEQ ID NO: 9
GASGSFSRPGLPVEYLQVPSPSMGRDIKVQFQSGGNNSPAVYLLDGLRAQ
DDYNGWDINTPAFEWYYQSGLSIVMPVGGQSSFYSDWYSPACGKAGCQTY
KWETFLTSELPQWLSANRAVKPTGSAAIGLSMAGSSAMILAAYHPQQFIY
AGSLSALLDPSQGMGPSLIGLAMGDAGGYKAADMWGPSSDPAWERNDPTQ
QIPKLVANNTRLWVYCGNGTPNELGGANIPAEFLENFVRSSNLKFQDAYN
AAGGHNAVFNFPPNGTHSWEYWGAQLNAMKGDLQSSLGAGGGGASGGAPG
GLNDIFEAQKIEWHE
depicts recombinant Ag85B nucleotide sequence
SEQ ID NO: 10
GGTGCTAGCGGCAGCTTCTCCCGGCCGGGGCTGCCGGTCGAGTACCTGCA
GGTGCCGTCGCCGTCGATGGGCCGCGACATCAAGGTTCAGTTCCAGAGCG
GTGGGAACAACTCACCTGCGGTTTATCTGCTCGACGGCCTGCGCGCCCAA
GACGACTACAACGGCTGGGATATCAACACCCCGGCGTTCGAGTGGTACTA
CCAGTCGGGACTGTCGATAGTCATGCCGGTCGGCGGGCAGTCCAGCTTCT
ACAGCGACTGGTACAGCCCGGCCTGCGGTAAGGCTGGCTGCCAGACTTAC
AAGTGGGAAACCTTCCTGACCAGCGAGCTGCCGCAATGGTTGTCCGCCAA
CAGGGCCGTGAAGCCCACCGGCAGCGCTGCAATCGGCTTGTCGATGGCCG
GCTCGTCGGCAATGATCTTGGCCGCCTACCACCCCCAGCAGTTCATCTAC
GCCGGCTCGCTGTCGGCCCTGCTGGACCCCTCTCAGGGGATGGGGCCTAG
CCTGATCGGCCTCGCGATGGGTGACGCCGGCGGTTACAAGGCCGCAGACA
TGTGGGGTCCCTCGAGTGACCCGGCATGGGAGCGCAACGACCCTACGCAG
CAGATCCCCAAGCTGGTCGCAAACAACACCCGGCTATGGGTTTATTGCGG
GAACGGCACCCCGAACGAGTTGGGCGGTGCCAACATACCCGCCGAGTTCT
TGGAGAACTTCGTTCGTAGCAGCAACCTGAAGTTCCAGGATGCGTACAAC
GCCGCGGGCGGGCACAACGCCGTGTTCAACTTCCCGCCCAACGGCACGCA
CAGCTGGGAGTACTGGGGCGCTCAGCTCAACGCCATGAAGGGTGACCTGC
AGAGTTCGTTAGGCGCCGGCGGTGGAGGCGCCTCAGGCGGCGCGCCTGG
AGGTCTGAACGACATCTTCGAGGCTCAGAAAATCGAATGGCACGAG
depicts BAP tag amino acid sequence
SEQ ID NO: 11
GLNDIFEAQKIEWHE

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a method of immobilizing polypeptides on a surface, said method comprising: (a) coating a surface with a molecule to capture biotin tagged polypeptide to obtain a coated surface; and (b) contacting at least two biotin tagged polypeptides with the coated surface of step (a) to obtain immobilized polypeptides, wherein the biotin tagged polypeptide comprises biotin linked to a recombinant polypeptide.

In an embodiment of the present disclosure, there is provided a method of immobilizing polypeptides on a surface described herein, wherein the molecule is selected from a group consisting of streptavidin, anti-biotin antibody, avidin, neutravidin, captavidin, and their derivatives.

In an embodiment of the present disclosure, there is provided a method of immobilizing polypeptides on a surface as described herein, wherein the recombinant polypeptide has a nucleic sequence as set forth in SEQ ID NO: 1. In another embodiment of the present disclosure, the recombinant polypeptide has a nucleic sequence as set forth in SEQ ID NO: 3. In yet another embodiment of the present disclosure, the recombinant polypeptide has a nucleic sequence as set forth in SEQ ID

NO: 5. In an alternate embodiment of the present disclosure, the recombinant polypeptide has a nucleic sequence as set forth in SEQ ID NO: 7. In still another embodiment of the present disclosure, the recombinant polypeptide has a nucleic sequence as set forth in SEQ ID NO: 9. It can be contemplated that any mixture comprising any combination of two or more than two recombinant polypeptides can be used for contacting with the coated surface of step (a). As per one embodiment, a mixture of recombinant polypeptides comprises polypeptides having an amino acid sequence as set forth in SEQ ID NO: 1, SEQ ID NO: 3, SEQ ID NO: 5, SEQ ID NO: 7, and SEQ ID NO: 9.

In an embodiment of the present disclosure, there is provided a method of immobilizing polypeptides on a surface as described herein, wherein contacting at least two biotin tagged polypeptides with the coated surface refers to contacting a mixture of individual polypeptides comprising the recombinant polypeptides having an amino acid sequence as set forth in SEQ ID NO: 1, SEQ ID NO: 3, SEQ ID NO: 5, SEQ ID

NO: 7, and SEQ ID NO: 9.

In an embodiment of the present disclosure, there is provided an in-vitro method for detecting at least one binder in a sample, said method comprising: (a) coating a surface with a molecule to capture biotin tagged polypeptide to obtain a coated surface; (b) contacting at least two biotin tagged polypeptides with the coated surface of step (a) to obtain immobilized polypeptides, wherein the biotin tagged polypeptide comprises a biotin linked to a recombinant polypeptide; (c) obtaining a sample; (d) adding the sample to the immobilized polypeptides to obtain a binder-polypeptide complex; (e) detecting the binder-polypeptide complex, wherein detecting the binder-polypeptide complex indicates the presence of at least one binder in the sample. In another embodiment, the binder is selected from a group consisting of antibodies, aptamers, affibodies and other non-antibody scaffolds. In yet another embodiment, the binder is antibody.

In an embodiment of the present disclosure, there is provided an in-vitro method for detecting at least one antibody in a sample, said method comprising: (a) coating a surface with a molecule to capture biotin tagged polypeptide to obtain a coated surface; (b) contacting at least two biotin tagged polypeptides with the coated surface of step (a) to obtain immobilized polypeptides, wherein the biotin tagged polypeptide comprises a biotin linked to a recombinant polypeptide; (c) obtaining a sample; (d) adding the sample to the immobilized polypeptides to obtain an antibody-polypeptide complex; and (e) detecting the antibody-polypeptide complex, wherein detecting the antibody-polypeptide complex indicates the presence of at least one antibody in the sample.

In an embodiment of the present disclosure, there is provided a method as described herein, wherein the surface is selected from a group consisting of glass, plastic, membrane, metal, and magnetic surface.

In an embodiment of the present disclosure, there is provided an in-vitro method of detecting at least one antibody as described herein, wherein the antibody is either a monoclonal antibody or a polyclonal antibody.

In an embodiment of the present disclosure there is provided a method as described herein, wherein the recombinant polypeptide has amino acid sequence selected from a group consisting of SEQ ID NO: 1, SEQ ID NO: 3, SEQ ID NO: 5, SEQ ID NO: 7, and SEQ ID NO: 9.

In an embodiment of the present disclosure there is provided a method as described herein, wherein the recombinant polypeptide has an amino acid sequence as set forth in SEQ ID NO: 1.

In an embodiment of the present disclosure there is provided a method as described herein, wherein the recombinant polypeptide has an amino acid sequence as set forth in SEQ ID NO: 3.

In an embodiment of the present disclosure there is provided a method as described herein, wherein the recombinant polypeptide has an amino acid sequence as set forth in SEQ ID NO: 5.

In an embodiment of the present disclosure there is provided a method as described herein, wherein the recombinant polypeptide has an amino acid sequence as set forth in SEQ ID NO: 7.

In an embodiment of the present disclosure there is provided a method as described herein, wherein the recombinant polypeptide has an amino acid sequence as set forth in SEQ ID NO: 9.

In an embodiment of the present disclosure there is provided a method as described herein, wherein biotin is linked to the recombinant polypeptide at either C-terminus or N-terminus. In another embodiment, biotin is linked to the recombinant polypeptide enzymatically by recombinant BirA enzyme.

In an embodiment of the present disclosure there is provided an in-vitro method of detecting at least one binder in a sample as described herein, wherein detecting is by a method selected form the group consisting of ELISA, lateral flow strip assay, biopanning selection assay, and color-coded bead-based assay.

In an embodiment of the present disclosure, there is provided an in-vitro method for detection of anti-Mycobacterium antibody in a sample, said method comprising: (a) coating a surface with a molecule to capture biotin tagged polypeptide to obtain a coated surface; (b) contacting at least two biotin tagged polypeptides with the coated surface of step (a) to obtain immobilized polypeptides, wherein the biotin tagged polypeptide comprises a biotin linked to a recombinant polypeptide, and the recombinant polypeptide has an amino acid sequence selected from a group consisting of SEQ ID NO: 1, SEQ ID NO: 3, SEQ ID NO: 5, SEQ ID NO: 7, and SEQ ID NO: 9; (c) obtaining a sample; (d) adding the sample to the immobilized polypeptides to obtain an antibody-polypeptide complex; and (e) detecting the antibody-polypeptide complex, wherein detecting the antibody-polypeptide complex indicates the presence of anti-Mycobacterium antibody in the sample.

In an embodiment of the present disclosure, there is provided an in-vitro method for detection of anti-Mycobacterium antibody in a sample as described herein, wherein the recombinant polypeptide has an amino acid sequence as set forth in SEQ ID NO: 1.

In an embodiment of the present disclosure, there is provided an in-vitro method for detection of anti-Mycobacterium antibody in a sample as described herein, wherein the recombinant polypeptide has an amino acid sequence as set forth in SEQ ID NO: 3.

In an embodiment of the present disclosure, there is provided an in-vitro method for detection of anti-Mycobacterium antibody in a sample as described herein, wherein the recombinant polypeptide has an amino acid sequence as set forth in SEQ ID NO: 5.

In an embodiment of the present disclosure, there is provided an in-vitro method for detection of anti-Mycobacterium antibody in a sample as described herein, wherein the recombinant polypeptide has an amino acid sequence as set forth in SEQ ID NO: 7.

In an embodiment of the present disclosure, there is provided an in-vitro method for detection of anti-Mycobacterium antibody in a sample as described herein, wherein the recombinant polypeptide has an amino acid sequence as set forth in SEQ ID NO: 9.

In an embodiment of the present disclosure, there is provided an in-vitro method for detection of anti-Mycobacterium antibody in a sample as described herein, wherein the at least two biotin tagged polypeptides for contacting with the coated surface is the recombinant polypeptide having an amino acid sequence as set forth in SEQ ID NO:1 and SEQ ID NO: 3. In another embodiment, the recombinant polypeptide has an amino acid sequence as set forth in SEQ ID NO: 1, SEQ ID NO: 3, and SEQ ID NO: 5 are contacted with the coated surface as a mixture. In yet another embodiment, the recombinant polypeptide has an amino acid sequence as set forth in SEQ ID NO: 1, SEQ ID NO: 3, SEQ ID NO: 5, and SEQ ID NO: 7 are contacted with the coated surface as a mixture. It can be contemplated that the at least two biotin tagged polypeptide refers to any combination of the recombinant polypeptide has an amino acid sequence as set forth in SEQ ID NO: 1, SEQ ID NO: 3, SEQ ID NO: 5, SEQ ID NO: 7, and SEQ ID NO: 9.

In an embodiment of the present disclosure, there is provided an in-vitro method for detection of anti-Mycobacterium antibody in a sample as described herein, wherein the at least two biotin tagged polypeptides for contacting with the coated surface refers to a polypeptide mixture comprising the recombinant polypeptide having an amino acid sequence as set forth in SEQ ID NO: 1, SEQ ID NO: 3, SEQ ID NO: 5, SEQ ID NO: 7, and SEQ ID NO: 9.

In an embodiment of the present disclosure, there is provided a biotin tagged polypeptide comprising a recombinant polypeptide linked to biotin, wherein the recombinant polypeptide having an amino acid sequence is selected from a group consisting of SEQ ID NO: 1, SEQ ID NO: 3, SEQ ID NO: 5, SEQ ID NO: 7, and SEQ ID NO: 9.

In an embodiment of the present disclosure, there is provided a biotin tagged polypeptide as described herein, wherein biotin is linked to the recombinant polypeptide at either C-terminus or N-terminus. In another embodiment, biotin is linked to the recombinant polypeptide enzymatically by recombinant BirA enzyme.

In an embodiment of the present disclosure, there is provided a recombinant nucleic acid molecule having nucleotide sequence selected from a group consisting of SEQ ID NO. 2, SEQ ID NO. 4, SEQ ID NO. 6, SEQ ID NO. 8, and SEQ ID NO. 10.

In an embodiment of the present disclosure, there is provided a recombinant vector comprising a recombinant nucleic acid molecule having nucleotide sequence selected from a group consisting of SEQ ID NO. 2, SEQ ID NO. 4, SEQ ID NO. 6, SEQ ID NO. 8, and SEQ ID NO. 10, operably linked to a promoter to drive the expression of the recombinant nucleic acid molecule.

In an embodiment of the present disclosure, there is provided a recombinant host cell comprising a vector, said vector comprising a recombinant nucleic acid molecule having nucleic acid sequence selected from the group consisting of SEQ ID NO. 2, SEQ ID NO. 4, SEQ ID NO. 6, SEQ ID NO. 8, and SEQ ID NO. 10, operably linked to a promoter to drive the expression of the recombinant nucleic acid molecule.

In an embodiment of the present disclosure, there is provided a recombinant host cell as described herein, wherein the host cell is selected from a group consisting of a bacterial cell, a fungal cell, a yeast cell, and mammalian cell lines. In another embodiment, the host cell is a bacterial cell.

In an embodiment of the present disclosure, there is provided a process for expression of a recombinant polypeptide, said process comprising the steps of: (a) obtaining a recombinant host cell comprising a vector, said vector comprising a recombinant nucleic acid molecule having nucleotide sequence selected from the group consisting of SEQ ID NO. 2, SEQ ID NO. 4, SEQ ID NO. 6, SEQ ID NO. 8, and SEQ ID NO. 10, operably linked to a promoter to drive the expression of the recombinant nucleic acid molecule; (b) growing the recombinant host cell in a growth medium under suitable conditions for the expression of the recombinant polypeptide.

In an embodiment of the present disclosure, there is provided a process of preparing a biotin tagged polypeptide, said process comprising the steps of: (a) obtaining a recombinant host cell comprising a vector, said vector comprising a recombinant nucleic acid molecule having nucleotide sequence selected from a group consisting of SEQ ID NO. 2, SEQ ID NO. 4, SEQ ID NO. 6, SEQ ID NO. 8, and SEQ ID NO. 10, operably linked to a promoter to drive the expression of the recombinant nucleic acid molecule; (b) growing the recombinant host cell in a growth medium under suitable conditions to express a recombinant polypeptide, wherein the recombinant polypeptide comprises a histidine affinity tag, a TEV protease site, and a BAP tag having amino acid sequence as depicted in SEQ ID NO: 11; (c) contacting the recombinant polypeptide of step (b) with an affinity chromatographic support; (d) eluting a polypeptide fraction 1 from the affinity chromatographic support; wherein the polypeptide fraction 1 has a histidine affinity tag; (e) contacting the polypeptide fraction 1 with a gel filtration chromatographic support; (f) eluting a polypeptide fraction 2 from the gel filtration chromatographic support, wherein the polypeptide fraction 2 has a histidine affinity tag; (g) treating the polypeptide fraction 2 with tagged TEV protease to remove the affinity tag from the polypeptide fraction 2 to obtain a polypeptide fraction 3; (h) contacting the polypeptide fraction 3 with an affinity chromatographic support; (i) eluting a polypeptide fraction 4 from the affinity chromatographic support, wherein the polypeptide fraction 4 does not have a histidine affinity tag; (j) contacting the polypeptide fraction 4 with an anion-exchange chromatographic support; (k) eluting the recombinant polypeptide from the anion-exchange chromatographic support; (l ) enzymatically linking the recombinant polypeptide of step (k) to a biotin molecule using histidine tagged recombinant BirA enzyme; and (m) removing the histidine tagged recombinant BirA enzyme from enzymatically linked recombinant polypeptide of step (l) by affinity chromatography to obtain the biotin tagged polypeptide.

In an embodiment of the present disclosure, there is provided a process of preparing a biotin tagged polypeptide as described herein, wherein said vector comprises a recombinant nucleic acid molecule having a nucleic acid sequence as set forth in SEQ ID NO. 2. In another embodiment of the present disclosure, said vector comprises a recombinant nucleic acid molecule having a nucleotide sequence as set forth in SEQ ID NO. 4 or SEQ ID NO: 6 or SEQ ID NO: 8 or SEQ ID NO: 10.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

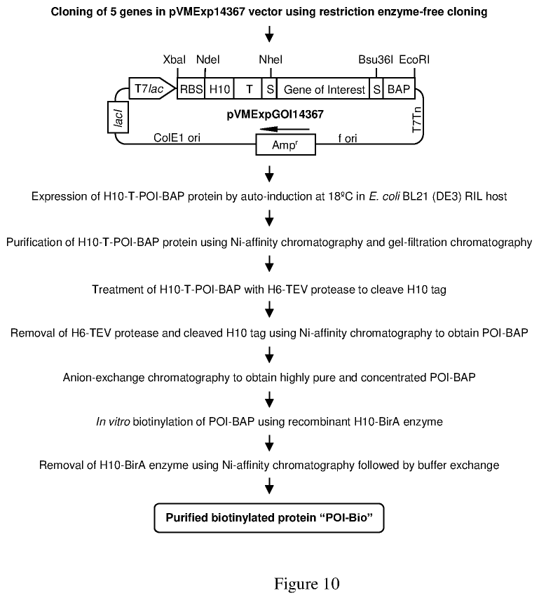

The subsequent paragraphs provide an optimized and streamlined process flow for cloning, high level expression and efficient purification to obtain specific biotin tagged protein that is devoid of any affinity tag. the cloning is performed using a newly designed vector that allows efficient and robust restriction-enzyme free cloning of desired genes. The purification involves a multi-step sequential process which majorly involves an affinity chromatographic support, a gel filtration chromatographic support, and an anion exchange chromatographic support. The purification steps further involve removal of the affinity tags from the proteins and enzymatic addition of biotin tag onto the C-terminus of the protein. Working examples, demonstrating application of such biotin tagged protein in detection of monoclonal and polyclonal antibodies in multiplexed ELISA has also been shown in the instant application. The biotin tagged proteins can also be used in other platforms, such as, in lateral flow tests. Further, detailed protocol for the experiments have been provided for MTC28, however, it is to be understood that same procedure will be followed for all the other proteins, i.e. MPT63, MPT64, Ag85A and Ag85B. FIG. 10 provides a simplified workflow for expression, purification, and in vitro biotinylation of five mycobacterial proteins.

### Material and Methods

Escherichia coli strains BL21 (DE3) RIL (B F⁻ ompT hsdS (r_{B}⁻ m_{B}⁻) dcm+Tet^{r} gall (DE3) endA Hte [argU ileY leuW Cam^{r}), and TOP10F′ (F′ [lacI^{q} Tn10 (tet^{R})] mcrA Δ(mrr-hsdRMS-mcrBC) φ80lacZΔM15 ΔlacX74 deoR nupG recA1 araD139 Δ(ara-leu)7697 galU galK rpsL(Str^{R}) endA1 λ⁻) were obtained from commercial sources.

Chemicals such as ATP (disodium salt) was obtained from Roche, Mannheim, Germany D-Biotin and all other standard chemicals were obtained from Affymetrix, Calif., USA.

HRP-conjugated Goat anti-Mouse IgG (H+L) antibody and HRP-conjugated Goat anti-Rabbit IgG (H+L) antibody were obtained from Jackson Immuno Research Laboratories Inc, PA, US. Oligonucleotides were obtained from Sigma-Aldrich, Bangalore, India. Restriction enzymes, T4 DNA ligase, and T4 DNA polymerase were obtained from NEB, Ipswich, Mass., USA. PfuUltra II Fusion HS DNA polymerase was obtained from Agilent Technologies, Santa Clara, US.

Nunc-Maxisorp polystyrene 384 well plates with clear, flat bottom (cat. no. 464718), Nunc Immobilizer streptavidin 384 well plates with clear, flat bottom, and covalently coated streptavidin (cat no. 436017), Chromatography resins and columns were obtained from GE Healthcare Life Sciences, Uppsala, Sweden. Reagents for polyacrylamide gel electrophoresis were obtained from Bio-Rad, Hercules, USA.

Recombinant hexa-histidine-tagged TEV protease (H6-TEV protease) carrying mutation S219V was expressed and purified in-house from vector pRK793 (obtained from Addgene; plasmid 8827), following protocols as described by Tropea et al (Methods in molecular biology, 2009, 498:297-307). Recombinant N-terminal deca-histidine-tagged E. coli BirA enzyme (H10-BirA) was produced in-house using a T7 promoter-lac operator-based expression vector, pVLExpBirA4231. The H10-BirA protein was purified from cytosolic fraction using a two-step protocol involving affinity chromatography on Ni Sepharose Fast Flow resin (NiFF), and gel-filtration chromatography on Superdex 200 to obtain tens of milligram of pure ˜38 kDa monomeric protein. Purified mouse monoclonal antibodies MTC28-13 (MTC28 specific), MPT63-03 (MPT63 specific), MPT64-33 (MPT64 specific), Ag85-14 (Ag85A specific), Ag85-11 (Ag85B specific), and Ag85-12 (Ag85A and Ag85B specific) derived from hybridoma clones were prepared in-house. Rabbit polyclonal sera against purified polyhistidine-tagged proteins (without BAP tag) were produced by a commercial source, Bangalore Genei, India (Merck), and purified in-house.

### Example 1

Construction of expression vector pVMExp14367: Vector pVMExp14367 is a medium copy number T7 promoter-lac operator-based expression vector containing sequence encoding N-terminal deca-histidine tag (H10), Tobacco Etch Virus (TEV) protease cleavage site, 2.0 Kb SacR-SacB gene cassette encoding levansucrase protein of Bacillus subtilis flanked by two appropriately oriented BsaI sites, and 15 amino acid C-terminal Biotin Acceptor Peptide tag (BAP). In between the functional tags/protease site, there are glycine-serine rich spacer sequences of appropriate lengths. The vector backbone comprises the ColE1 origin of replication (ori) with deletion of rop gene, filamentous phage origin of replication (f ori), β-lactamase gene as a selection marker and lac repressor (lacI). This vector design is compatible with highly efficient restriction enzyme-free cloning of the genes (Chaudhary et. al. PLoS One, 2014; 9(10):e111538). The vector pVMExp14367 was constructed by assembly of several components encoding T7 promoter-lac operator, H10 tag, TEV protease cleavage site, ampicillin resistance marker; beta-lactamase gene, and ColE1 ori from a set of intermediate vectors, and synthetic DNA sequences available in the laboratory. SacR-SacB gene cassette was obtained as a synthetic gene from Geneart (Thermo Fisher Scientific, Waltham, US), and BAP tag was assembled using duplex of oligonucleotides encoding 15 amino acid sequence. The final recombinant was sequenced using ABI 3730 XL DNA sequencing platform (Applied Biosystems, Thermo Fisher Scientific, Waltham, USA). The GenBank accession number of the vector pVMExp14367 is MG599491.

### Example 2

Cloning of M. tuberculosis genes in the expression vector pVMExp14367: The protocol used for cloning genes in vector pVMExp14367, as obtained in Example 1, is as described in “Rapid restriction enzyme-free cloning of PCR products: a high-throughput method applicable for library construction” (Chaudhary et. al. PLoS One, 2014; 9(10):e111538). Briefly, DNA encoding five M. tuberculosis H37Rv proteins viz. MTC28 (Rv0040c), MPT63 (Rv1926c), MPT64 (Rv1980c), Ag85A (Rv3804c), and Ag85B (Rv1886c) was amplified from respective templates (as depicted in Table 1) below, using 5′ and 3′ gene specific primers carrying 7 base extensions to append sequences required for restriction enzyme-free cloning. SEQ ID NO: 12 to 21 refers to the respective primer sequence as disclosed in Table 1 below.

TABLE 1

| Gene of | Size | | Primers | |
|---|---|---|---|---|
| Interest | (bp) | Template for PCR | Name | Sequence (5′ - 3′) |
| Ag85A | 899 bp | pVLExpAg85A4337 | Ag85A-5-1 | CGGCAGCTTTTCCCGGCCGGGCTTGCCGGTG |
| (Rv3804c) | | | (SEQ ID NO: 12) | |
| | | | Ag85A-N3-1 | CTCCACCGGCGCCCTGGGGCGCGGGCCCGGT |
| | | | (SEQ ID NO: 13) | |
| Ag85B | 869 bp | pVMExpAg85B4231 | Ag85B-5-1 | CGGCAGCTTCTCCCGGCCGGGGCTGCCGGTC |
| (Rv1886c) | | | (SEQ ID NO: 14) | |
| | | | Ag85B-N3-1 | CTCCACCGCCGGCGCCTAACGAACTCTGCAG |
| | | | (SEQ ID NO: 15) | |
| MPT63 | 404 bp | pVMExpMPT634231 | MPT63-5-1 | CGGCAGCGCCTATCCCATCACCGGAAAACTT |
| (Rv1926c) | | | (SEQ ID NO: 16) | |
| | | | MPT63-N3-1 | CTCCACCCGGCTCCCAAATCAGCAGATCCTC |
| | | | (SEQ ID NO: 17) | |
| MPT64 | 629 bp | pVMExpMPT644231 | MPT64-5-1 | CGGCAGCGCGCCCAAGACCTACTGCGAGGAG |
| (Rv1980c) | | | (SEQ ID NO: 18) | |
| | | | MPT64-N3-1 | CTCCACCGGCCAGCATCGAGTCGATCGCGGA |
| | | | (SEQ ID NO: 19) | |
| MTC28 | 848 bp | pVMExpMTC284337 | MTC28-51 | CGGCAGCGATCCCCTGCTGCCACCGCCGCCTATC |
| (Rv0040c) | | | (SEQ ID NO: 20) | |
| | | | MTC28-31 | CTCCACCGCGCGGCGGGACTGGTGTCAGGGT |
| | | | (SEQ ID NO: 21) | |

For cloning, the inserts were prepared using “column method”. “Column method” refers to the restriction-enzyme free method for cloning of PCR products amplified using high-fidelity polymerases as described in Chaudhary et. al. PLoS One, 2014; 9(10):e111538. The BsaI-digested linearized vector was also prepared. Ligation reaction was set up in 10 μl reaction volume containing 1.0 μl of the T4 DNA polymerase-treated insert (˜50 ng), 50 ng BsaI-digested linearized and purified pVMExp14367 vector, and 200 U of T4 DNA Ligase (400 U/μl) in 1× ligation buffer (well known in the art). The ligation reaction was carried out at 16° C. for 1 hr and 37° C. for 1 hr followed by inactivation of T4 DNA ligase at 65° C. for 10 min The ligation reaction (1 μl) was diluted to 10 μl in distilled water, and 500 pg equivalent ligated vector was electroporated in 25 μl electrocompetent E. coli BL21 (DE3) RIL cells (electroporation efficiency ˜5×10⁸/μg pGEM DNA), and plated on MDAG plates containing 100 μg/ml ampicillin and 30 μg/ml chloramphenicol (MDAGAmp₁₀₀Cm₃₀). Three transformants of each construct were checked by sequencing on ABI 3730 XL sequencing platform (Applied Biosystems, Thermo Fisher Scientific, Waltham, USA), and clones with correct sequence were chosen for expression studies.

### Example 3

Expression and Localization of the proteins: Small-scale expression and localization was carried out to determine the yield of soluble protein. Protein expression was performed by auto-induction in 50 ml ZYM5052 media. The clones from MDAGAmp₁₀₀Cm₃₀ plates (from Example 2) were inoculated in 3 ml MDAGAmp₁₀₀Cm₃₀ liquid media (primary culture) and grown at 30° C. for 18 hr. The primary culture was diluted 100-fold in 50 ml ZYM5052 media containing 100 μg/ml ampicillin and 30 μg/ml chloramphenicol (ZYM5052Amp₁₀₀Cm₃₀) and grown with shaking at 250 rpm at 30° C. for 2 hr, 24° C. for 4 hr, and 18° C. for 16 hr. The cells were harvested and re-suspended in 25 ml of 1×TLB (50 mM Tris-HCl, pH 7.5 buffer containing 500 mM NaCl) supplemented with lysozyme at a final concentration of 200 μg/ml and PMSF at a final concentration of 0.1 mM, and incubated on ice for 30 min. Cells were lysed using sonication. The lysate was centrifuged at 22,000 g for 30 min at 4° C. to obtain the supernatant (HSS; High Speed Supernatant), which was further centrifuged at 50,000 g for 2 hr at 4° C., and the resulting supernatant containing soluble proteins was named as HHSS (High-High Speed Supernatant). For localization of the recombinant proteins, different sub-cellular fractions including HSS and HHSS were analyzed on 0.1% SD-8-20% polyacrylamide gradient gel (PAG) under reducing conditions. Based on the yield of soluble recombinant proteins in HHSS as determined by Coomassie brilliant blue R-250 dye stained PAG, an appropriate volume of preparative scale culture was set up for all the five proteins under the same expression conditions as described above. After auto-induction, the cells were harvested and resuspended in half the volume of 1×TLB (without the addition of lysozyme and PMSF), followed by homogenization of cells using PANDA high-pressure homogenizer (GEA Niro Soavi) at 800 bars for 6 cycles as per the manufacturer's instructions. The lysate was centrifuged, and HHSS was prepared as described for the small-scale expression. The HHSS fraction containing soluble proteins was then subjected to three-step chromatography protocol to obtain purified protein preparations.

### Example 4

Purification of proteins: The entire purification procedure was performed at 4-8° C. using appropriate chromatography columns attached to the AKTA Explorer 100 system (GE Healthcare Life Sciences, Uppsala, Sweden). During chromatography, the eluted protein was monitored using absorbance at 280 nm; fractions (of appropriate volumes depending on the type of chromatography) were collected and analyzed using SDS-PAGE under reducing conditions. Based on the purity and yield of the proteins, fractions were pooled.

For purification of H10-T-MTC28-BAP (Histidine Tag-TEV protease site-Protein of interest-BAP Tag) 280 ml HHSS containing approximately 336 mg of recombinant protein in 1×TLB with 20 mM imidazole was filtered through 0.45μ membrane, and applied on 20 ml Ni Sepharose Fast Flow (NiFF) resin packed in HR16/10 column (pre-equilibrated with 1×TLB containing 20 mM imidazole) at a flow rate of 3 ml/min. After loading of the sample, the column was washed with 60 ml (3 CV) of 1×TLB containing 20 mM imidazole, followed by 160 ml (8 CV) of 1×TLB containing 50 mM imidazole at a flow rate of 3.5 ml/min Finally, the bound protein was eluted with 1×TLB containing 300 mM imidazole at 2 ml/min and 2 ml fractions were collected. The fractions were analyzed using 0.1% SDS-12.5% PAGE and those containing desired protein were pooled (NiFF pool).

The NiFF pool was further purified by gel-filtration chromatography on 480 ml Superdex 75 column pre-equilibrated with 20 mM Tris-HCl, pH 8.0 containing 50 mM NaCl. The NiFF pool was loaded at a flow rate of 3 ml/min, the column was subsequently developed at a flow rate of 3 ml/min, and 4 ml fractions were collected. The fractions were analyzed using 0.1% SDS-12.5% PAGE and those containing relatively pure protein were pooled (GFC pool).

Next, to obtain tagless protein preparation (i.e. MTC28-BAP from H10-T-MTC28-BAP), the purified protein in the GFC pool was subjected to treatment with H6-TEV protease. For this, approximately 198 mg of H10-T-MTC28-BAP was subjected to digestion with 2 mg H6-TEV protease (1:100 ratio of TEV protease:substrate; w/w) in a reaction volume of 45 ml in 1×TEV reaction buffer (50 mM Tris-HCl, pH 8.0, 0.5 mM EDTA, 1 mM DTT). The reaction was gently mixed by inversion, centrifuged and incubated at 30° C. for 3 hr. After completion of the reaction, sample equivalent to ˜8 μg protein was analyzed on 0.1% SDS-12.5% PAGE to determine the extent of cleavage. After completion of the digestion process, 5 ml of 10× loading supplement (2.5 M NaCl, 10 mM MgCl₂, 50 mM Tris-HCl pH 8.0, 200 mM imidazole) was added to the reaction. The resultant digestion mixture containing 20 mM imidazole in 1×TLB, was applied on a 20 ml NiFF resin packed in HR16/10 column (pre-equilibrated with 1×TLB containing 20 mM imidazole) at a flow rate of 1 ml/min. This allowed the H6-TEV protease, the cleaved H10 tag, and the uncleaved protein (if any) to bind to the column, whereas the protein of interest i.e. tagless MTC28-BAP was collected in the flow-through fractions. These fractions were pooled as NiFF pool after TEV protease Treatment (NiFF-TT pool).

Finally, the NiFF-TT pool containing tagless protein was subjected to anion-exchange chromatography. For this, the NiFF-TT pool was desalted using Sephadex G25 (140 ml resin packed in XK 26/40 column), pre-equilibrated with 20 mM Tris-HCl, pH 8.0 (buffer A). For desalting the entire NiFF-TT pool (˜60 ml), the same column was used twice (30 ml sample was desalted per run) with column re-equilibration in between the two runs. For anion-exchange chromatography, the desalted NiFF-TT pool (˜80 ml) was applied on 20 ml Q Sepharose High Performance (QHP) resin (packed in HR16/10 column; pre-equilibrated with buffer A) at a flow rate of 4 ml/min The QHP column was washed with 60 ml (3 CV) of buffer A, elution was performed with 100 ml (5 CV) linear gradient of 0-0.5 M NaCl in buffer A at 4 ml/min, and 2 ml fractions were collected. Fractions were analyzed using 0.1% SDS-12.5% PAGE and fractions containing pure protein were pooled (named as QHP pool). The protein concentration of QHP pool was estimated by measuring absorbance at 280 nm The remaining four proteins were also purified employing the same process as described above for MTC28-BAP to obtain purified tagless proteins.

### Example 5

In vitro biotinylation of purified tagless proteins using E. coli H10-BirA enzyme: For biotinylation of the proteins, the QHP pool containing purified C-terminal BAP-tagged protein was subjected to in vitro biotinylation using recombinant deca-histidine-tagged BirA enzyme (H10-BirA). For each protein, approximately 10 mg of the QHP purified pool (˜2 ml) was subjected to buffer-exchange using 13 ml Sephadex G25 (fine) syringe column equilibrated in 50 mM Tris-HCl, pH 8.0 buffer. The protein eluted in approximately 4 ml volume. The in vitro biotinylation reaction was set up in a 6-ml volume containing 10 mg of purified C-terminal BAP-tagged protein (substrate), with 10 mM ATP, 10 mM MgCl₂, 50 μM d-biotin, and 0.2 mg of recombinant H10-BirA enzyme (to achieve enzyme to substrate ratio of 1:50, w/w), and incubated at 30° C. for 3 hr. After completion, 660 μl of 10× loading supplement (2.5 M NaCl, 10 mM MgCl₂, 50 mM Tris-HCl pH 8.0, 200 mM imidazole) was added to the reaction and applied on 1.2 ml NiFF column at a flow rate of 0.2 ml/min and 0.5 ml flow-through fractions were collected. This allowed binding of H10-BirA enzyme to the column, and the biotinylated protein collected in the flow-through fractions. The flow-through fractions were pooled, and final volume was reduced to 2 ml using amicon-ultra-4 centrifugal filter unit (10 kDa Cut Off; Merck Millipore) followed by desalting on 13 ml Sephadex G25 (fine) syringe column in 1×PBS buffer (20 mM phosphate, pH 7.5 containing 150 mM NaCl) to obtain purified tagless and C-terminal biotinylated proteins. The protein concentration was estimated by measuring absorbance at 280 nm. Purified biotinylated proteins were analyzed using 0.1% SDS-12.5% PAGE under reducing conditions.

### Example 6

Determination of the extent of biotinylation in biotin-tagged proteins: To determine the extent of biotinylation, 25 μg of each biotin-tagged protein was adsorbed on 50 μl Streptavidin Sepharose HP beads (taken in 3-5 folds excess based on the binding capacity to ensure complete adsorption) by constant mixing for 1 hr at 25° C. in a 2 ml tube (click cap, clear, round bottom microtubes, Treff Lab, Switzerland). The supernatant (labelled as ‘after’ adsorption fraction) was collected after centrifugation of mixture at 12,000 g for 5 min at 25° C. Separately, a similarly diluted sample of each biotin-tagged protein was prepared and labeled as ‘before’ adsorption fraction. To determine the amount of protein remaining after adsorption, both ‘before’ and ‘after’ adsorption fractions were tested using indirect ELISA with protein-specific monoclonal antibodies to determine the amount of protein. For this, 384-well Nunc Immobilizer streptavidin-coated plate was washed thrice with PBST (1×PBS with 0.05% Tween-20), and coated with 25 μl each of 7 point 3-fold dilutions of ‘before’ (range ˜1:100-1:100K) and ‘after’ (range ˜1:10-1:10K) adsorption fractions (prepared in 1×PBS) for 2 hr at 25° C. Wells were washed thrice with PBST and blocked with 2% BSA-PBST for 1 hr at 25° C. After blocking, plate was washed thrice with PBST and proteins were probed with 100 ng/ml of respective protein-specific monoclonal antibodies (MAb MTC28-13 for MTC28, MAb MPT63-03 for MPT63, MAb MPT64-33 for MPT64, and MAb Ag85-12 for Ag85A and Ag85B) diluted in 0.1% BSA-PBST for 1 hr at 25° C. The plate was washed thrice with PBST, and HRP-conjugated Goat anti-Mouse IgG (H+L) antibody diluted 1:5000 times in 0.1% BSA-PBST was added for 1 hr at 25° C. Finally, after three washes each with PBST and 1×PBS, the reaction was revealed by 25 μl TMB substrate (Seramun Diagnostics, Berlin, Germany) Following, incubation in the dark for 15 min at 25° C., the reaction was terminated by addition of 25 μl 1 N H2504 and absorbance was measured at 450 nm using ELISA plate reader (SpectraMax M5; Molecular Devices, Sunnyvale, Calif., USA). The extent of biotinylation was calculated based on the fold reduction in protein reactivity after adsorption on streptavidin beads.

### Example 7

Indirect ELISA with biotin-tagged antigens using mouse monoclonal or rabbit polyclonal antibodies: To compare the coating efficiency of biotinylated proteins individually, different dilutions of biotin-tagged proteins (7 point 3-fold dilutions; range ˜1 μg/ml to ˜1.3 ng/ml) were prepared in 1×PBS, and 25 μl of each was added to either 384 well Nunc Immobilizer streptavidin-coated plate (pre-washed thrice with PBST) for 2 hr at 25° C., or 384 well Nunc Maxisorp polystyrene plate for 2 hr at 37° C. Wells were washed thrice with PBST and blocked with 2% BSA-PBST for 1 hr at 25° C. After blocking, the plates were washed thrice with PBST and the proteins were probed either with 100 ng/ml of protein-specific purified mouse monoclonal antibodies (MAb MTC28-13 for MTC28, MAb MPT63-03 for MPT63, MAb MPT64-33 for MPT64, MAb Ag85-14 for Ag85A, and MAb Ag85-11 for Ag85B), or with 100 ng/ml of protein-specific purified rabbit polyclonal antibodies (both diluted in 0.1% BSA-PBST) for 1 hr at 25° C. Following this, the plates were washed thrice with PBST, and HRP-conjugated Goat anti-Mouse IgG (H+L) (diluted 1:5000 times in 0.1% BSA-PBST), or HRP-conjugated Goat anti-Rabbit IgG (H+L) antibodies (diluted 1:10,000 times in 0.1% BSA-PBST) were added for 1 hr at 25° C. to probe the bound mouse monoclonal or rabbit polyclonal antibodies, respectively. Remaining steps were performed as described above. To compare the coating efficiency of a mixture of biotin-tagged proteins, 10 μg each of 5 antigens (viz. biotinylated MTC28, MPT63, MPT64, Ag85A and Ag85B) was mixed (i.e. equal ratio, w/w), and 7 point 3-fold dilutions of resultant mixture (range ˜5 μg/ml to ˜7 ng/ml of the mixture, equivalent to ˜1 μg/ml to ˜1.3 ng/ml of individual protein) were captured on 384 well Nunc Immobilizer streptavidin-coated plate (pre-washed thrice with PBST) for 2 hr at 25° C., or 384 well Nunc Maxisorp polystyrene plate for 2 hr at 37° C. Remaining steps were performed as described above.

### Results

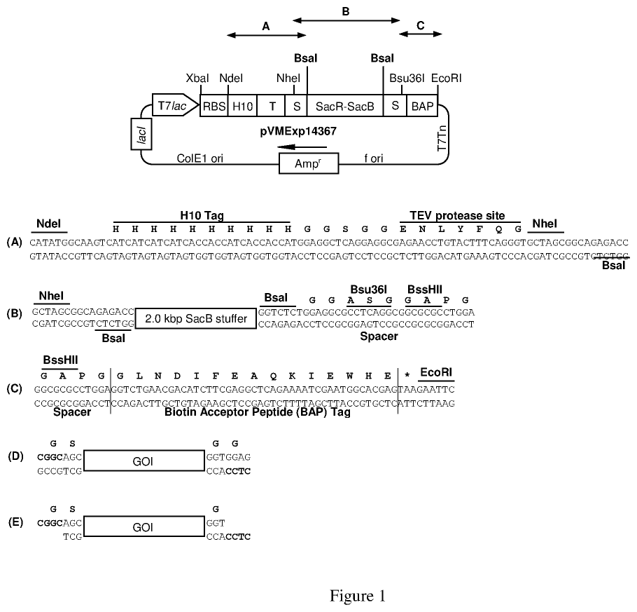

Construction of the vector pVMExp14367 and cloning of genes: A T7 promoter-lac operator-based IPTG/lactose inducible vector pVMExp14367 was constructed for expression of recombinant proteins in E. coli carrying N-terminal deca-histidine tag (H10) followed by TEV protease site and C-terminal Biotin Acceptor Peptide (BAP) tag with appropriate glycine-serine rich spacers as depicted in FIG. 1. This format allows for affinity-based purification of the expressed recombinant proteins using the H10 tag, and its subsequent specific removal by the virtue of TEV protease site to obtain proteins devoid of the N-terminal tag. The C-terminal BAP tag allows for in vitro site-specific biotinylation of the proteins using recombinant E. coli BirA enzyme. The DNA encoding proteins for MTC28 (Rv0040c), MPT63 (Rv1926c), MPT64 (Rv1980c), Ag85A (Rv3804c), and Ag85B (Rv1886c) (without signal sequence) was cloned in the vector pVMExp14367 (as described in Example 2) using restriction enzyme-free cloning strategy. In this strategy, the vector is prepared by digestion with Type IIs restriction enzyme BsaI, whose two recognition sites are present in the vector flanking the stuffer in two orientations (FIG. 1B) in a manner that allows generation of 4 base 5′-overhangs. The insert for cloning is prepared by PCR amplification using primers carrying 20-23 base long gene-specific sequence with 7 base long additional sequence (5′-CGGCACC-3′ in forward primer and 5′-

CTCCACC-3′ in reverse primer). The resulting insert is then subjected to treatment with T4 DNA polymerase in the presence of dTTP to generate desired 4 base 5′-overhangs compatible with the vector (shown in bold; FIGS. 1D and 1E). Transformants in E. coli host BL21 (DE3) RIL were screened by sequencing and 100% cloning efficiency was observed.

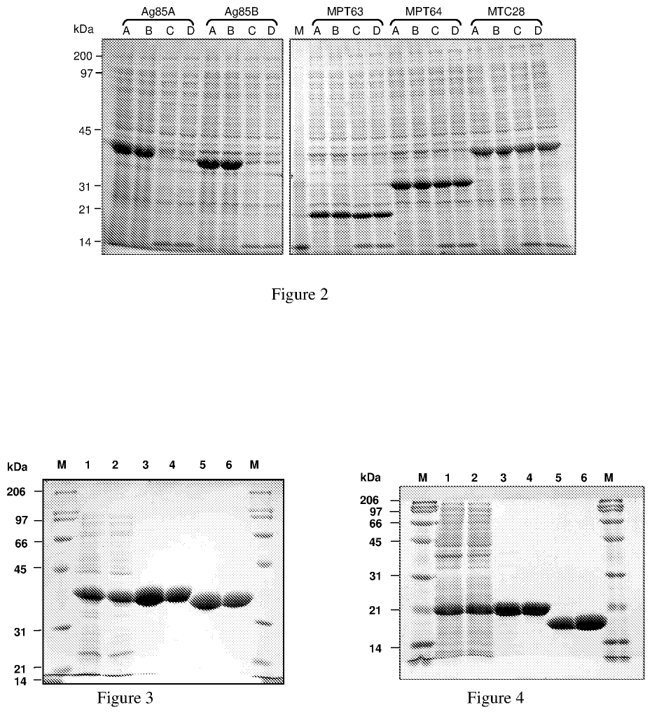

Expression and localization of the proteins: Protein expression was performed using auto-induction process in ZYM5052 media (described in Example 3) at low-temperature conditions that promote solubility of the proteins. As can be observed in FIG. 2, all the five proteins showed good expression in total cell fractions (Lane A), and constituted ˜15-30% of the total cellular protein. Lane B and Lane C refer to total cell fraction after sonication and fraction after HSS, respectively. The yield of soluble protein in the HHSS cytosolic fraction varied among different proteins (Lane D). MTC28, MPT63, and MPT64 proteins were nearly 100% soluble, with almost the entire fraction of expressed protein present in the HHSS fraction, whereas the amount of Ag85A and Ag85B proteins in the HHSS fraction was only ˜5% of the total expressed protein (Lane D). Based on the yield of the soluble proteins, appropriate volumes of auto-induced cultures were processed (Table 2; Column 1), and the 2×HHSS fraction containing soluble protein was subjected to purification.

Purification of the proteins: The recombinant proteins carried H10 tag followed by TEV protease site at the N-terminus and BAP tag at the C-terminus (i.e. H10-T-POI-BAP; POI-Protein of interest). To purify proteins, a streamlined workflow was developed, which involved two-step purification of the proteins including affinity chromatography and gel-filtration chromatography. The purified monomeric/dimeric fraction of the proteins obtained after gel-filtration chromatography was then subjected to treatment with H6-tagged TEV protease to obtain proteins devoid of the N-terminus H10 tag, followed by removal of TEV protease and cleaved tag from the purified protein preparations using Ni-affinity chromatography. The proteins (devoid of H10-tag) were finally purified using anion-exchange chromatography and then subjected to in vitro biotinylation using H10-BirA enzyme and purification of the fully biotinylated protein.

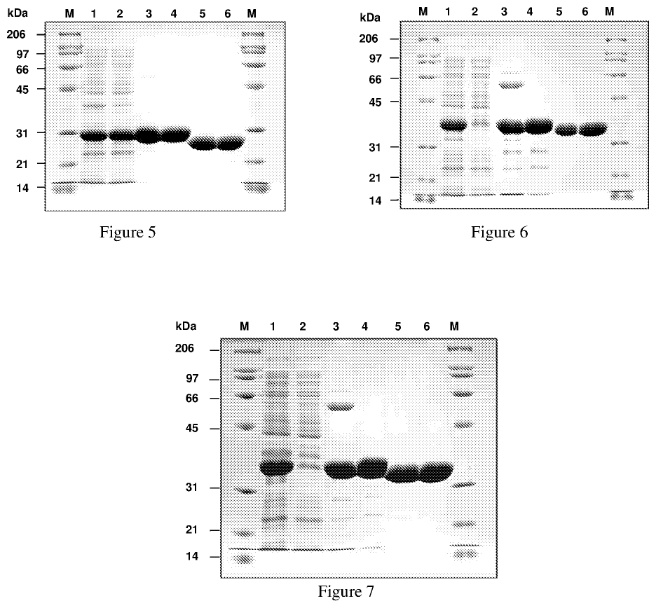

FIG. 3, FIG. 4, FIG. 5, FIG. 6, and FIG. 7 depict SDS gel images of the proteins MTC28, MPT63, MPT64, Ag85A, and Ag85B, respectively. The summary of the lanes of the gel is as depicted in Table 2 below.

TABLE 2

| | |
|---|---|
| Lane 1 | Total cell after homogenization |
| Lane 2 | High High Speed Supernatant (HHSS) |
| Lane 3 | Pool after NiFF (affinity) chromatography |
| Lane 4 | Pool after gel filtration chromatography |
| Lane 5 | Pool after H10 tag removal followed by |
| | NiFF (affinity) chromatography |
| Lane 6 | Pool after ion exchange chromatography. |
| Lane M | molecular weight marker |

Yield of each of the protein at sequential chromatographic stages corresponding to SDS gel images have been depicted in Table 3 below.

TABLE 3

| | | | | | | Yield after |
|---|---|---|---|---|---|---|
| | | | Yield after | | | anion |
| | Volume of | | Ni-affinity | Yield after | | exchange |
| | culture | Amount of | chromatog | TEV protease | Yield after | chromato |
| Protein | (OD₆₀₀ₙₘ) | POI in HHSS^{a} | raphy^{b,c} | removal^{c} | desalting^{f,c} | graphy^{g} |
| (H10-T-POI-BAP) | (1) | (2) | (3) | (5) | (6) | (7) |
| MTC28 | 560 ml (9.8) | ~330 mg/280 ml | ~300 mg/24 ml | ~180 mg/60 ml | ~150 mg/80 ml | ~112 mg/12 ml |
| MPT63 | 560 ml (8.9) | ~330 mg/280 ml | ~140 mg/26 ml | ~170 mg/69 ml | ~150 mg/96 ml | ~120 mg/12 ml |
| MPT64 | 560 ml (9.2) | ~330 mg/280 ml | ~190 mg/26 ml | ~255 mg/66 ml | ~225 mg/88 ml | ~150 mg/12 ml |
| Ag85A | 2000 ml (11.1) | ~100 mg/1000 ml | ~90 mg/28 ml | ~75 mg/66 ml | ~55 mg/83 ml | ~41 mg/14 ml |
| Ag85B | 2000 ml (9.5) | ~120 mg/1000 ml | ~90 mg/28 ml | ~90 mg/70 ml | ~70 mg/88 ml | ~42 mg/10 ml |

It is apparent from FIGS. 3-7 and Table 3, that all the proteins obtained were highly pure. A good yield of greater than 40 mg/10 ml was also observed.

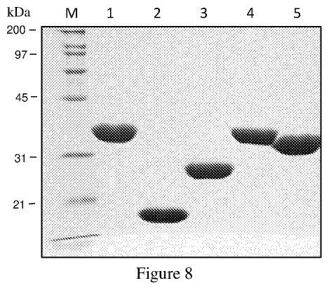

In vitro biotinylation of the purified proteins using H10-BirA enzyme: In vitro biotinylation of the proteins were performed as described in Example 5. FIG. 8 depicts the SDS gel image of the biotinylated proteins Lane 1, lane 2, lane 3, lane 4 and lane 5 refer to biotinylated MTC28, MPT63, MPT64, Ag85A, and Ag85B respectively. The image reveals that integrity of proteins was maintained during biotinylation process.

Determination of the extent of biotinylation in biotin-tagged proteins: As a measure of the efficiency of the in vitro biotinylation reaction, the extent of biotinylation in biotin-tagged proteins was determined as described in Example 6. Based on the fold reduction in the reactivity of fractions after adsorption on streptavidin beads (a measure of remaining protein amount), the extent of biotinylation was determined and was found to be greater than 99.5% for all the five biotin-tagged proteins (Table 4). This indicated that in vitro biotinylation reaction using recombinant H10-BirA enzyme was highly efficient.

TABLE 4

| | Fold dilution to achieve | | Fold | |
|---|---|---|---|---|
| | OD_{450 nm} ~0.5 ^{a} | | reduction in | |
| | Before | After | protein | |
| | adsorption on | adsorption on | reactivity | |
| | streptavidin | streptavidin | after | Extent of |
| Protein | beads | beads | adsorption | biotinylation |
| MTC28- | 25,000 | 50 | 500 | 99.8% |
| Bio | | | | |
| MPT63- | 100,000 | 100 | 1000 | 99.9% |
| Bio | | | | |
| MPT64- | 25,000 | 50 | 500 | 99.8% |
| Bio | | | | |
| Ag85A- | 20,000 | 5 | 4000 | 99.9% |
| Bio | | | | |
| Ag85B- | 30,000 | 30 | 1000 | 99.9% |
| Bio | | | | |

Fixed amount of biotin-tagged proteins was adsorbed on Streptavidin Sepharose HP beads. This was followed by estimation of protein amount in ‘before’ and ‘after’ adsorption fractions using indirect ELISA on Nunc Immobilizer streptavidin-coated plates, where the proteins were probed with specific monoclonal antibodies followed by detection using HRP-conjugated Goat anti-Mouse IgG (H+L) antibody. The values are based on the mean of two independent experiments.

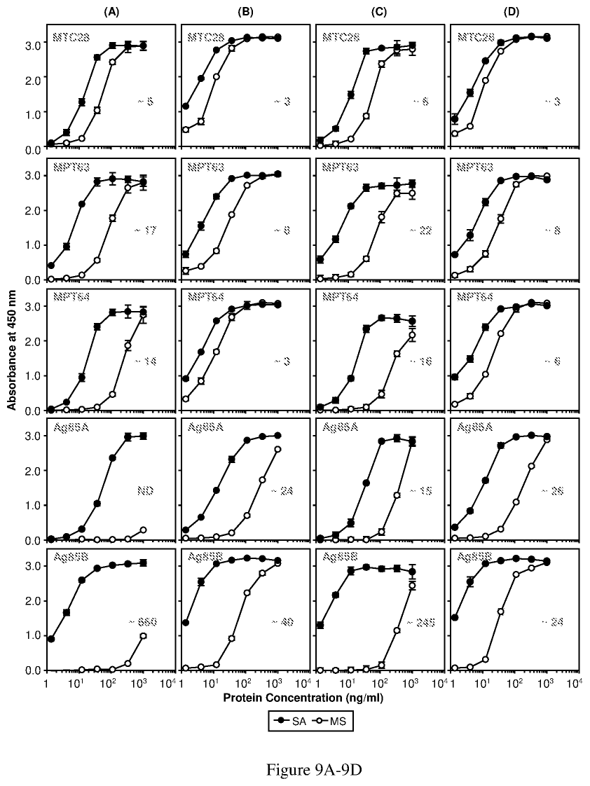

Indirect ELISA with biotin-tagged antigens using mouse monoclonal or rabbit polyclonal antibodies: To evaluate the performance of biotin-tagged proteins, ELISA using immobilization by passive adsorption on the polystyrene surface, and specific capture on the streptavidin-coated surface was performed as described in Example 7. For this purpose, different concentrations of biotin-tagged proteins were coated on the two types of microtiter plate surfaces, and the captured proteins were detected using indirect ELISA with either specific mouse monoclonal antibodies, or rabbit polyclonal antibodies. In this assay, the amount of protein added for coating on the two surfaces to produce equal reactivity was compared. When detected with monoclonal antibodies, the assay required approximately 5-660-fold less protein to produce comparable signal in ELISA upon specific capture using streptavidin-coated plates, which is due to the efficient capture of biotin-tagged proteins on the streptavidin-coated surface (FIG. 9A). This was especially evident in the case of Ag85A-Bio and Ag85B-Bio proteins, where for passive adsorption, much higher protein concentration (greater than 1000 ng/ml) was required to produce a significant signal (A_{450 nm} of ˜1.0) in comparison to MTC28-Bio, MPT63-Bio, and MPT64-Bio proteins, which were required in much lower concentrations (approximately 100-200 ng/ml) (FIG. 9A). This suggested that Ag85A-Bio and Ag85B-Bio proteins tend to coat poorly on the polystyrene surface during passive adsorption. However, upon specific capture on the streptavidin-coated plates, both Ag85A-Bio and Ag85B-Bio proteins produced a significant signal at lower protein concentrations (approximately 1-30 ng/ml), which was comparable to other biotin-tagged proteins (approximately 1-10 ng/ml) (FIG. 9A).

When the detection in ELISA was performed using polyclonal antibodies, efficient protein-coating was observed upon specific capture on the streptavidin-coated plates (FIG. 9B). However, the difference between specific and passive immobilization was less pronounced (approximately 3-40-fold) here as compared to the assay performed using monoclonal antibodies (FIG. 9A versus FIG. 9B). This difference could likely be due to the higher affinity and polyclonal nature of the rabbit antibodies. The monoclonal antibodies bind to only one site on the protein (the epitope), and the same may not be accessible on every passively coated molecule, whereas, the polyclonal antibodies bind to multiple epitopes on the same molecule, and thus, even upon passive coating, a larger number of epitopes could be accessible. In contrast, on streptavidin-coated plates, all the epitopes are likely to be exposed, except for those, which are present at the C-terminus of the protein; therefore, even monoclonal antibodies show significant binding.

To mimic the assay conditions, where the coating of proteins as a mixture may be necessary, we also compared the efficiency of specific and passive immobilization of each protein in a mixture. In this case also, the specific immobilization resulted in reduced requirement of proteins to produce comparable reactivity in ELISA upon detection both mouse monoclonal antibodies (˜6-245-fold), and rabbit polyclonal antibodies (˜3-26 fold) (FIGS. 9C and 9D).

This property of efficient capture of biotinylated proteins using streptavidin-coated plates can be used as an effective strategy to improve the ELISA sensitivity, particularly for the proteins like Ag85A and Ag85B that exhibit poor coating characteristics upon passive immobilization. This finding is particularly relevant for developing ELISA-based assays for antibody detection, where it might be necessary to coat multiple proteins with variable surface binding characteristics.

Depicted in FIG. 10, is a simplified flowchart illustrating the steps in the preparation of the biotin tagged polypeptides.

Advantage of the present disclosure: The present invention provides a streamlined strategy for easy and robust cloning, high-level soluble expression, and efficient purification to obtain highly pure tagless proteins at large scale, along with their subsequent in vitro biotinylation for the production of biotin-tagged proteins. The efficiency of the biotin-tagged proteins has been demonstrated in multiplexed ELISA. The results showed that on the streptavidin-coated surface, much lower protein concentrations were required to produce a significant signal in ELISA as compared to passive adsorption (FIGS. 9A and 9B). Another important finding was that on streptavidin-coated plates, every protein was captured almost uniformly while, in conventional coating technique through passive adsorption, every protein showed different coating behaviour. This uniformity in the capture of biotin-tagged proteins on the streptavidin-coated surfaces is particularly significant for developing ELISA-based assays for antibody detection, where it might be necessary to coat multiple proteins as a mixture, and uniformity of coating cannot be ensured due to variable coating properties of individual proteins present in the mixture. The biotin tagged proteins can also be used to develop assays such as lateral flow strip assay and biopanning assay. In summary, the streamlined strategy with efficient and robust protocols described here will accelerate the production of biotinylated proteins to facilitate several applications including the development of multiplexed immunoassays for antibody detection in patient sera.

I/We claim:

1. A method of immobilizing polypeptides on a surface, said method comprising:
a) coating a surface with a molecule to capture biotin tagged polypeptide to obtain a coated surface; and
b) contacting at least two biotin tagged polypeptides with the coated surface of step (a) to obtain immobilized polypeptides,
wherein the biotin tagged polypeptide comprises biotin linked to a recombinant polypeptide.

2. An in-vitro method for detecting at least one binder in a sample, said method comprising:
a) coating a surface with a molecule to capture biotin tagged polypeptide to obtain a coated surface;
b) contacting at least two biotin tagged polypeptides with the coated surface of step (a) to obtain immobilized polypeptides, wherein the biotin tagged polypeptide comprises biotin linked to a recombinant polypeptide;
c) obtaining a sample;
d) adding the sample to the immobilized polypeptides to obtain a binder-polypeptide complex; and
e) detecting the binder-polypeptide complex,
wherein detecting the binder-polypeptide complex indicates the presence of at least one binder in the sample.

3. An in-vitro method for detecting at least one antibody in a sample, said method comprising:
a) coating a surface with a molecule to capture biotin tagged polypeptide to obtain a coated surface;
b) contacting at least two biotin tagged polypeptides with the coated surface of step (a) to obtain immobilized polypeptides, wherein the biotin tagged polypeptide comprises biotin linked to a recombinant polypeptide;
c) obtaining a sample;
d) adding the sample to the immobilized polypeptides to obtain an antibody-polypeptide complex; and
e) detecting the antibody-polypeptide complex,
wherein detecting the antibody-polypeptide complex indicates the presence of at least one antibody in the sample.

4. The method as claimed in any one of the claims 1-3, wherein the surface is selected from a group consisting of glass, plastic, membrane, metal, and magnetic surface.

5. The method as claimed in any one of the claims 1-3, wherein the molecule is selected from a group consisting of streptavidin, anti-biotin antibody, avidin, neutravidin, captavidin, and their derivatives.

6. The method as claimed in any one of the claims 1-3, wherein the recombinant polypeptide has amino acid sequence selected from a group consisting of SEQ ID NO: 1, SEQ ID NO: 3, SEQ ID NO: 5, SEQ ID NO: 7, and SEQ ID NO: 9.

7. The method as claimed in any one of the claims 1-3, wherein biotin is linked to the recombinant polypeptide at either C-terminus or N-terminus.

8. The method as claimed in claim 7, wherein biotin is linked to the recombinant polypeptide enzymatically by recombinant BirA enzyme.

9. The method as claimed in any one of the claims 2-3, wherein detecting is by a method selected from a group consisting of Enzyme-linked Immunosorbent Assay (ELISA), lateral flow strip assay, color-coded bead-based assay and biopanning selection assay.

10. An in-vitro method for detection of anti-Mycobacterium antibody in a sample, said method comprising:
a) coating a surface with a molecule to capture biotin tagged polypeptides to obtain a coated surface;
b) contacting at least two biotin tagged polypeptides with the coated surface of step (a) to obtain immobilized polypeptides, wherein the biotin tagged polypeptide comprises biotin linked to a recombinant polypeptide, and the recombinant polypeptide has amino acid sequence selected from a group consisting of SEQ ID NO: 1, SEQ ID NO: 3, SEQ ID NO: 5, SEQ ID NO: 7, and SEQ ID NO: 9;
c) obtaining a sample;
d) adding the sample to the immobilized polypeptides to obtain an antibody-polypeptide complex; and
e) detecting the antibody-polypeptide complex,
wherein detecting the antibody-polypeptide complex indicates the presence of anti-Mycobacterium antibody in the sample.

11. A biotin tagged polypeptide comprising a recombinant polypeptide linked to biotin, wherein the recombinant polypeptide has an amino acid sequence selected from a group consisting of SEQ ID NO: 1, SEQ ID NO: 3, SEQ ID NO: 5, SEQ ID NO: 7, and SEQ ID NO: 9.

12. The biotin tagged polypeptide as claimed in claim 11, wherein biotin is linked to the recombinant polypeptide at either C-terminus or N-terminus.

13. The biotin tagged polypeptide as claimed in claim 12, wherein biotin is linked to the recombinant polypeptide enzymatically by recombinant BirA enzyme.

14. A recombinant nucleic acid molecule having nucleotide sequence selected from a group consisting of SEQ ID NO. 2, SEQ ID NO. 4, SEQ ID NO. 6, SEQ ID NO. 8, and SEQ ID NO. 10.

15. A recombinant vector comprising the recombinant nucleic acid molecule as claimed in claim 14, operably linked to a promoter to drive the expression of the recombinant nucleic acid molecule.

16. A recombinant host cell comprising the vector as claimed in claim 15.

17. The recombinant host cell as claimed in claim 16, wherein the host cell is selected from a group consisting of a bacterial cell, a fungal cell, a yeast cell, and mammalian cell lines.

18. The recombinant host cell as claimed in claim 17, wherein the host cell is a bacterial cell.

19. A process for expression of a recombinant polypeptide, said process comprising the steps of:
a) obtaining a recombinant host cell as claimed in any one of the claims 16-18; and
b) growing the recombinant host cell in a growth medium under suitable conditions for the expression of the recombinant polypeptide.

20. A process of preparing a biotin tagged polypeptide as claimed in claim 11, said process comprising the steps of:
a) obtaining a recombinant host cell as claimed in any one of the claims 16-18;
b) growing the recombinant host cell in a growth medium under suitable conditions to express a recombinant polypeptide, wherein the recombinant polypeptide comprises a histidine affinity tag, a TEV protease site, and a BAP tag having amino acid sequence as depicted in SEQ ID NO: 11;
c) contacting the recombinant polypeptide of step (b) with an affinity chromatographic support;
d) eluting a polypeptide fraction 1 from the affinity chromatographic support; wherein the polypeptide fraction 1 has a histidine affinity tag;
e) contacting the polypeptide fraction 1 with a gel filtration chromatographic support;
f) eluting a polypeptide fraction 2 from the gel filtration chromatographic support, wherein the polypeptide fraction 2 has a histidine affinity tag;
g) treating the polypeptide fraction 2 with tagged TEV protease to remove the affinity tag from the polypeptide fraction 2 to obtain a polypeptide fraction 3;
h) contacting the polypeptide fraction 3 with an affinity chromatographic support;
i) eluting a polypeptide fraction 4 from the affinity chromatographic support, wherein the polypeptide fraction 4 does not have a histidine affinity tag;
j) contacting the polypeptide fraction 4 with an anion-exchange chromatographic support;
k) eluting the recombinant polypeptide from the anion-exchange chromatographic support,
l) enzymatically linking the recombinant polypeptide of step (k) to a biotin molecule using histidine tagged recombinant BirA enzyme; and
m) removing the histidine tagged recombinant BirA enzyme from enzymatically linked recombinant polypeptide of step (l) by affinity chromatography to obtain the biotin tagged polypeptide.


SEQUENCE LISTING (21 sequences)

SEQ ID NO: 1 - protein, 308 aa, Artificial Sequence
  SEQ ID NO 1 depicts recombinant MTC28 amino acid sequence
GASGSDPLLPPPPIPAPVSAPATVPPVQNLTALPGGSSNRFSPAPAPAPIASPIPVGAPGSTAVPPLPPPVTPAISGTLRDHLREKGVKLEAQRPHGFKALDITLPMPPRWTQVPDPNVPDAFVVIADRLGNSVYTSNAQLVVYRLIGDFDPAEAITHGYIDSQKLLAWQTTNASMANFDGFPSSIIEGTYRENDMTLNTSRRHVIATSGADKYLVSLSVTTALSQAVTDGPATDAIVNGFQVVAHAAPAQAPAPAPGSAPVGLPGQAPGYPPAGTLTPVPPRGGGASGGAPGGLNDIFEAQKIEWHE

SEQ ID NO: 2 - DNA, 924 bp, Artificial Sequence
  SEQ ID NO 2 depicts recombinant MTC28 nucelic acid sequence
ggtgctagcggcagcgatcccctgctgccaccgccgcctatccctgccccagtctcggcgccggcaacagtcccgcccgtgcagaacctcacggcgcttccgggcgggagcagcaacaggttctcaccggcgccagcacccgcaccgatcgcgtcgccgattccggtcggagcacccgggtccaccgctgtgcccccgctgccgccgccagtgactcccgcgatcagcggcacacttcgggaccacctccgggagaagggcgtcaagctggaggcacagcgaccgcacggattcaaggcgctcgacatcacactgcccatgccgccgcgctggactcaggtgcccgaccccaacgtgcccgacgcgttcgtggtgatcgccgaccggttgggcaacagcgtctacacgtcgaatgcgcagctggtagtgtataggctgatcggtgacttcgatcccgctgaggccatcacacacggctacattgacagccagaaattgctcgcatggcagaccacaaacgcctcgatggccaatttcgacggctttccgtcatcaatcatcgagggcacctaccgcgaaaacgacatgaccctcaacacctcccggcgccacgtcatcgccacctccggagccgacaagtacctggtttcgctgtcggtgaccaccgcgctgtcgcaggcggtcaccgacgggccggccaccgatgcgattgtcaacggattccaagtggttgcgcatgcggcgcccgctcaggcgcctgccccggcacccggttcggcaccggtgggactacccgggcaggcgcctgggtatccgcccgcgggcaccctgacaccagtcccgccgcgcggtggaggcgcctcaggcggcgcgcctggaggtctgaacgacatcttcgaggctcagaaaatcgaatggcacgag

SEQ ID NO: 3 - protein, 160 aa, Artificial Sequence
  SEQ ID NO 3 depicts recombinant MPT63 amino acid sequence
GASGSAYPITGKLGSELTMTDTVGQVVLGWKVSDLKSSTAVIPGYPVAGQVWEATATVNAIRGSVTPAVSQFNARTADGINYRVLWQAAGPDTISGATIPQGEQSTGKIYFDVTGPSPTIVAMNNGMEDLLIWEPGGGASGGAPGGLNDIFEAQKIEWHE

SEQ ID NO: 4 - DNA, 480 bp, Artificial Sequence
  SEQ ID NO 4 depicts recombinant MPT63 nucleotide sequence
ggtgctagcggcagcgcctatcccatcaccggaaaacttggcagtgagctaacgatgaccgacaccgttggccaagtcgtgctcggctggaaggtcagtgatctcaaatccagcacggcagtcatccccggctatccggtggccggccaggtctgggaggccactgccacggtcaatgcgattcgcggcagcgtcacgcccgcggtctcgcagttcaatgcccgcaccgccgacggcatcaactaccgggtgctgtggcaagccgcgggccccgacaccattagcggagccactatcccccaaggcgaacaatcgaccggcaaaatctacttcgatgtcaccggcccatcgccaaccatcgtcgcgatgaacaacggcatggaggatctgctgatttgggagccgggtggaggcgcctcaggcggcgcgcctggaggtctgaacgacatcttcgaggctcagaaaatcgaatggcacgag

SEQ ID NO: 5 - protein, 235 aa, Artificial Sequence
  SEQ ID NO 5 depicts recombinant MPT64 amino acid sequence
GASGSAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGGGASGGAPGGLNDIFEAQKIEWHE

SEQ ID NO: 6 - DNA, 705 bp, Artificial Sequence
  SEQ ID NO 6 depicts recombinant MPT64 nucleotide sequence
ggtgctagcggcagcgcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggccggtggaggcgcctcaggcggcgcgcctggaggtctgaacgacatcttcgaggctcagaaaatcgaatggcacgag

SEQ ID NO: 7 - protein, 325 aa, Artificial Sequence
  SEQ ID NO 7 depicts recombinant Ag85A amino acid sequence
GASGSFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGGGASGGAPGGLNDIFEAQKIEWHE

SEQ ID NO: 8 - DNA, 975 bp, Artificial Sequence
  SEQ ID NO 8 depicts recombinant Ag85A nucleotide sequence
ggtgctagcggcagcttttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgccggtggaggcgcctcaggcggcgcgcctggaggtctgaacgacatcttcgaggctcagaaaatcgaatggcacgag

SEQ ID NO: 9 - protein, 315 aa, Artificial Sequence
  SEQ ID NO 9 depicts recombinant Ag85B amino acid sequence
GASGSFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGGGGASGGAPGGLNDIFEAQKIEWHE

SEQ ID NO: 10 - DNA, 945 bp, Artificial Sequence
  SEQ ID NO 10 depicts recombinant Ag85B nucleotide sequence
ggtgctagcggcagcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggcggtggaggcgcctcaggcggcgcgcctggaggtctgaacgacatcttcgaggctcagaaaatcgaatggcacgag

SEQ ID NO: 11 - protein, 15 aa, Artificial Sequence
  SEQ ID NO 11 depicts BAP tag amino acid sequence
GLNDIFEAQKIEWHE

SEQ ID NO: 12 - DNA, 31 bp, Artificial Sequence
  SEQ ID NO 12 depicts the primer sequence Ag85A-5-1
cggcagcttttcccggccgggcttgccggtg

SEQ ID NO: 13 - DNA, 31 bp, Artificial Sequence
  SEQ ID NO 13 depicts the primer sequence Ag85A-N3-1
ctccaccggcgccctggggcgcgggcccggt

SEQ ID NO: 14 - DNA, 31 bp, Artificial Sequence
  SEQ ID NO 14 depicts the primer sequence Ag85B-5-1
cggcagcttctcccggccggggctgccggtc

SEQ ID NO: 15 - DNA, 31 bp, Artificial Sequence
  SEQ ID NO 15 depicts the primr sequence Ag85B-N3-1
ctccaccgccggcgcctaacgaactctgcag

SEQ ID NO: 16 - DNA, 31 bp, Artificial Sequence
  SEQ ID NO 16 depicts the primer sequence MPT63-5-1
cggcagcgcctatcccatcaccggaaaactt

SEQ ID NO: 17 - DNA, 31 bp, Artificial Sequence
  SEQ ID NO 17 depicts the primer sequence MPT63-N3-1
ctccacccggctcccaaatcagcagatcctc

SEQ ID NO: 18 - DNA, 31 bp, Artificial Sequence
  SEQ ID NO 18 depicts the primer sequence MPT64-5-1
cggcagcgcgcccaagacctactgcgaggag

SEQ ID NO: 19 - DNA, 31 bp, Artificial Sequence
  SEQ ID NO 19 depicts the primer sequence MPT64-N3-1
ctccaccggccagcatcgagtcgatcgcgga

SEQ ID NO: 20 - DNA, 34 bp, Artificial Sequence
  SEQ ID NO 20 depicts the primer sequence MTC28-51
cggcagcgatcccctgctgccaccgccgcctatc

SEQ ID NO: 21 - DNA, 31 bp, Artificial Sequence
  SEQ ID NO 21 depicts the primer sequence MTC28-31
ctccaccgcgcggcgggactggtgtcagggt